(19) United States
(12) Patent Application Publication
(10) Pub. No.: US 2025/0095781 A1
(43) Pub. Date: Mar. 20, 2025

(54) METAGENOMIC FILTERING FOR DETECTING ALLERGEN AND TOXIGENS IN A FOOD PRODUCTION LINE

(71) Applicant: Mars, Incorporated, McLean, VA (US)

(72) Inventors: Balasubramanian GANESAN, Vancouver, WA (US); Robert C. BAKER, Geylang (SG)

(73) Assignee: Mars, Incorporated, McLean, VA (US)

(21) Appl. No.: 18/729,840

(22) PCT Filed: Jan. 9, 2023
(86) PCT No.: PCT/US2023/060298

§ 371 Date: Jul. 17, 2024

Related U.S. Application Data
(60) Provisional application No. 63/300,882, filed on Jan. 19, 2022.

(51) Int. Cl. G16B 30/10; G16B 35/10

(52) U.S. Cl. CPC G16B 30/10; G16B 35/10

(57) ABSTRACT

Methods for detecting an allergen or toxigen are disclosed herein. The methods comprise obtaining sequence data for a plurality of nucleic acid sequences present in a food product; identifying one or more allergen sequences, or one or more toxigen sequences, in the sequence data, wherein the one or more allergen sequences, or one or more toxigen sequences correspond to an allergen or a toxigen present in the food sample; and detecting the presence of the allergen or the toxigen in the food product if the one or more allergen sequences, or the one or more toxigen sequences, are above a predetermined threshold.

### CROSS REFERENCE TO RELATED APPLICATIONS

This application claims priority to U.S. Provisional Patent Application No. 63/300,882, filed Jan. 19, 2022, the contents of which are incorporated herein by reference in their entirety.

### FIELD OF THE DISCLOSURE

This disclosure relates to methods of detecting an allergen or toxigen in a food product using metagenomics filtering that is applied to food processing and manufacturing lines for detection and managing the outcomes.

### BACKGROUND OF THE DISCLOSURE

The presence of allergens and toxigens in food represents a major food safety issue. Food allergic reactions and other food hypersensitivities affect millions of people worldwide. Ingestion of food containing allergens can lead to severe adverse responses in an allergic individual, including general discomfort, dermatitis, asthma, and anaphylactic shock. Similarly, ingestion of toxigens can be potentially harmful to human health and can lead to food poisoning and even death. Early detection of allergens or toxigens in food products is therefore important for ensuring food safety and in protecting consumers.

Efficient and reliable techniques for detecting allergens and toxigens are required. Some existing allergen and toxigen detecting methods have relied on biochemical techniques, such as ELISA, PCR, or fluorescence- or chemiluminescence-based directed detection approaches. However, these methods are generally low-throughput, relying on analysis of individual allergens or toxigen, and generally cannot be performed using a single sample. Moreover, these methods are generally not compatible for combination with other food analytical methods, such as sequencing-based food authentication methods, and cannot be easily integrated into a food production chain trace-back system.

Accordingly, there is a need for high-throughput end-to-end methods for nucleic-acid-based detection of allergens and/or toxigens in food product.

### SUMMARY OF THE DISCLOSURE

Disclosed herein are methods of detecting an allergen or toxigen in a food product.

In one aspect, disclosed herein is a method for detecting the presence of an allergen in a food production chain, comprising: obtaining sequence data for a plurality of nucleic acid sequences present in a food product; identifying one or more allergen sequences in the sequence data, wherein the one or more allergen sequences correspond to an allergen present in the food sample; and detecting the presence of the allergen in the food product if the one or more allergen sequences are above a predetermined threshold. The allergen may be of milk or egg origin, for example.

In another aspect, disclosed herein is a method for detecting the presence of a toxigen in a food production chain, comprising: obtaining sequence data for a plurality of nucleic acid sequences present in a food product; identifying one or more toxigen sequences in the sequence data, wherein the one or more toxigen sequences correspond to a toxigen present in the food sample; and detecting the presence of an allergen in the food product if the one or more toxigen sequences are above a predetermined threshold. The toxigen may be a toxigen produced by bacteria, fungi or plants.

The predetermined threshold may correspond to the relative level of a sequence in the food sample. The predetermined threshold may also correspond to the sequence coverage of a sequence in the food sample.

The step of obtaining the sequence data may include preparing a sequencing library. Additionally, obtaining the sequence data may include next generation sequencing, or microarray analysis. The plurality of nucleic acid sequences are DNA or RNA sequences. The RNA sequences may correspond to mRNAs encoding polypeptides present in the food sample. These RNA sequences may be converted into amino acid sequences corresponding to polypeptides encoded by the RNA sequences prior to identifying the one or more allergen sequences or the one or more toxigen sequences.

In some instances, the sequences corresponding to the food product may be filtered from the sequence data prior to identifying the one or more allergen sequences or the one or more toxigen sequences. Sequences corresponding to microbes present in the product may also be filtered from the sequence data prior to identifying the one or more allergen sequences or the one or more toxigen sequences.

Identifying the one or more allergens may include comparing the sequence data against one or more databases of allergen sequences. Similarly, identifying the one or more toxigen sequences may include comparing the sequence data against one or more databases of toxigen sequences. The one or more databases of allergen sequences may correspond to databases of allergen protein sequences, while the one or more databases of toxigen sequences may correspond to databases of toxigen protein sequences.

Any of the methods provided herein may be performed at one or more points in a food production chain of the food product. The methods may also further include tracing the allergen or the toxigen to a particular supplier of the food product.

In some instances, some of the methods for detecting an allergen may further comprise identifying the food product as allergenic if the allergen is detected in the food sample and adjusting the product label information for the food product to indicate that the food product is allergenic.

In some instances, some of the methods for detecting a toxigen may further comprise identifying the food product as toxigenic if the toxigen is detected in the food sample and removing the food product from the food production chain if the product is identified as toxigenic.

In yet another aspect, provided herein is a system for detecting the presence of an allergen in a food production chain, comprising: one or more processors; and a memory comprising instructions executable by the one or more processors that, when executed by the one or more processors, cause the system to: obtain sequence data for a plurality of nucleic acid sequences present in a food product; identify one or more allergen sequences in the sequence data, wherein the one or more allergen sequences correspond to an allergen present in the food sample; and detect the presence of the allergen in the food product if the one or more allergen sequences are above a predetermined threshold. The memory may further comprise instructions executable by the one or more processors that, when executed by the one or more processors, cause the system to identify the food product as allergenic if the allergen is detected in the food sample, and adjust the product label information for the food product to indicate that the food product is allergenic.

In yet another aspect, provided herein is a system for detecting the presence of a toxigen in a food production chain, comprising: one or more processors; and a memory comprising instructions executable by the one or more processors that, when executed by the one or more processors, cause the system to: obtain sequence data for a plurality of nucleic acid sequences present in a food product; identify one or more toxigen sequences in the sequence data, wherein the one or more toxigen sequences correspond to a toxigen present in the food sample; and detect the presence of a toxigen in the food product if the one or more toxigen sequences are above a predetermined threshold. The memory may further comprise instructions executable by the one or more processors that, when executed by the one or more processors, cause the system to identify the food product as toxigenic if the toxigen is detected in the food sample, and prompt removal of the food product from the food production chain if the product is identified as toxigenic.

### BRIEF DESCRIPTION OF THE FIGURES

FIG. 1 is a flow diagram depicting a method of detecting an allergen or a toxigen in a food product.

FIG. 2 is a flow diagram depicting an exemplary data analysis process for the detection of allergen or toxigen in a food product. The dashed line indicates that allergen or toxigen identification can be performed directly using a database of nucleic acid sequences corresponding to particular allergens or toxigens.

FIG. 3 is an exemplary decision tree for detection of an allergen or toxigen in a food sample.

FIG. 4 is a decision tree for a working example of allergen detection in a food product using iterative logic for source identification.

FIG. 5 is a decision tree for a working example of allergen detection in a food product using parallel logic for source identification.

FIG. 6 is an exemplary heatmap depicting allergens detected in a food product. Each column represents a different food sample, while each row represents a different allergen. Allergens present in the food product are indicated by filled boxes, while white boxes correspond to allergens not detected in the food product.

FIG. 7 is an exemplary heatmap depicting the relative levels of allergens in a food product. Each column represents a different food sample, while each row represents a different allergen.

FIG. 8 is a heatmap depicting the relative quantification of allergens in a food product using a multi-color scheme and splitting trees to allow for source tracking. Each column represents a different food sample, while each row represents a different allergen.

FIG. 9 shows the distribution of allergens in different food matrices.

### DETAILED DESCRIPTION OF THE DISCLOSURE

The following description sets forth exemplary methods, conditions, and the like and are not intended as limiting the scope of the present disclosure. Instead, it is provided as a description of exemplary embodiments.

### I. Overview

Disclosed herein are methods and systems for detecting an allergen or a toxigen in a food product. The methods and systems disclosed herein are based on the analysis of allergen or toxigen sequences present in a food sample. Sequences corresponding to an allergen or a toxigen present over a pre-determined threshold may be used to detect the presence of said antigen or toxigen in a food product or food production line. The source of the allergen or toxigen can then be traced back to a particular raw material or supply chain.

Although the following description uses terms first, second, etc., to describe various elements, these elements should not be limited by the terms. These terms are only used to distinguish one element from another.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, rational numbers, steps, operations, elements, and/or components, but do not preclude the presence or addition of one or more other features, integers, rational numbers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “allergen” refers to any protein or protein fragment thereof, or any mixture of proteins that are known to induce an allergic response, e.g., an IgE-mediated immune response, in an individual, e.g., a human.

As used herein, the term “toxigen” refers to a protein, or protein fragment thereof, that is produced as a result of the bioactivity of a living cell or organism.

Metagenomics generally relates to the study of genetic material that is obtained from an environment (e.g. a food product or food factory surface or food processing equipment surface) and allows for analysis of a sample without the need to isolate the genetic material from individual species present in the sample. Metagenomics allows food samples to be analyzed in an unbiased, high throughput, and comprehensive manner. Moreover, metagenomics allows for specific sequences, such as sequences corresponding to food raw materials, to be removed prior to detection of allergen or toxigen sequences in a food product.

The methods and systems disclosed herein provide for detection of an allergen and/or a toxigen in a food production line. The methods described herein rely on the detection of allergens or toxigens in food products based on the identification of allergen or toxigen sequences that are present in the food products. In some instances, the detection of the allergens or toxigens is based on the identification of allergen or toxigen sequences that are present in the food products above a pre-determined threshold. Sequence data from nucleic acids present in food samples are analyzed to identify one or more allergen or toxigen sequences. The allergen or toxigen sequences can represent a subset of the metagenomics data obtained for a particular food product. The allergens or toxigens can be detected in a targeted manner by analyzing the data to identify sequences corresponding to a particular set of allergens or toxigens of interest, such as allergens or toxigens of high concern. Alternatively, the sequence data can be analyzed in an unbiased manner to identify allergen or toxigen sequences corresponding to any and all allergens or toxigens present in the food sample. The identification of allergen or toxigen sequences and their use to detect the presence of allergens or toxigens in a food source may not be dependent on the sequence data corresponding to the food raw materials themselves.

Furthermore, the methods disclosed herein may be used to trace-back the source of an allergen and/or a toxigen detected in a food production line to a particular supplier once the allergen and/or toxigen has been detected. For instance, if a particular allergen is detected in a food product, the testing producer can trace the allergen to a particular supplier. Upon traced back of the allergen to a supplier, the producer may seek corrective action or look to a different supplier. The producer can later use the methods disclosed herein to certify that the allergen is no longer present in the food production line.

Accordingly, the methods and system allow for improved accuracy and reliability in detecting allergens and/or toxigens in food products.

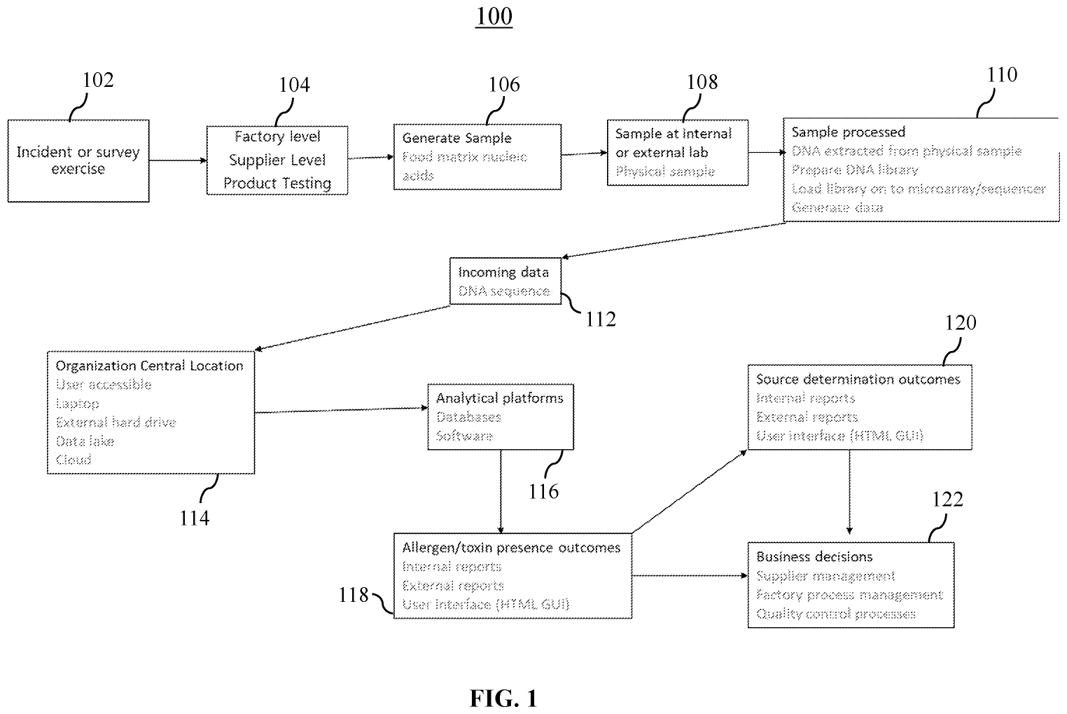

FIGS. 1-5 provide exemplary embodiments of methods for detecting an allergen or a toxigen in a food product, wherein sequence data for a food product is used to identify allergen or toxigen sequences that can be used to determine whether an allergen or a toxigen is present in the food product.

FIG. 1 depicts an exemplary flowchart of a method 100 for allergen or toxigen detection. At 102, the process may be initiated in response to an incident or survey exercise. The incident or survey exercise may be implemented as part of a regular monitoring process during any point of the food production line or implemented to detect allergens or toxigens in a raw material received from a new supplier. At 104, the incident or survey exercise prompts the implementation of the authentication method at, for example, the factory level, at the supplier level, or for product testing purposes. The method can be implemented at one or more particular points during each level of the food production chain. For instance, the method can be implemented at one or more of a transport step, a storage step, a processing step (e.g., a step involving pasteurization, cooling, mixing, grinding, marinating, boiling, melting, steaming, fermenting, etc.) or a packaging step (e.g., a step involving bottling, vacuum packaging, etc.) in the production chain of the food product. The method may also be implemented on the food product, or any of the raw materials used in the production of the food product. Moreover, at each level in the food production chain, one or more food products may be evaluated.

At 106, samples are generated from the food product at the designated testing level. The sample generation may involve preparation of food matrix nucleic acids. The sample is prepared at a designated location, such as an internal or external laboratory (108). Once the physical sample is received by the internal or external laboratory, the physical sample is processed at step 110. During sample processing, nucleic acids (e.g. DNA or RNA) are extracted from the physical sample, a sequencing library (e.g. a DNA library) is prepared, the library is analyzed (e.g. by loading the library onto a microarray or sequencer), and data is generated. Any known method for nucleic acid extraction and library preparation known in the art may be used. For instance, without limitation, nucleic acid extraction may be performed on freshly collected or frozen samples, and using any available extraction technique such as phenol: chloroform: isoamyl alcohol extraction or by using any appropriate commercially available kit. The sequencing library may be analyzed using any available technique that provides nucleic acid sequence data, such as, without limitation, next generation sequencing, qPCR, mass spectrometry, chromatography, microarray, in situ sequencing, probe hybridization, and any combination thereof. The sequencing library preparation will depend on the analysis technique to be used and can be prepared according to the manufacturer's instructions.

The generated data is transferred at 112 as incoming data (e.g., DNA sequence data) to a central location in the organization (114). The central location may be user accessible, such as a laptop, an external hard drive, a data lake or a cloud, or any other local or centralized system in the organization, or a data storage location available as a service to the organization. The transferred data may be provided from an internal laboratory or an external source and may be stored in the central location until further downstream analysis. At 116, the data from the central location is accessed by analytical platforms. The analytical platform would comprise one or more databases or software that would enable analysis of the sequence data. Analysis of the nucleic acid sequences can include, without limitation, comparing sequences against one or more databases; filtering sequence reads by size, quality, or origin; de-multiplexing a sample; sequence mapping; read quantification; or any combination thereof. Any suitable analytical platform, such as a platform comprising publicly available software or database, or in-house software or databases may be used.

Analysis of the data using the analytical platforms results in one or more antigen or toxigen presence outcomes (118). The one or more antigen or toxigen presence outcomes may be included in internal or external reports, which may be reported as a physical report, or displayed in a user interface. For instance, a user interface may display the one or more antigen or toxigen presence outcomes and allow a user to navigate and refine the outcomes (e.g., select a specific a particular set of outcomes). Additionally, the user interface may allow a user to compare one or more antigen or toxigen presence outcomes corresponding to different food products, different lots of the same food products, the same product lot at different lots in the food production chain, or any combination thereof.

The antigen or toxigen outcomes may be further analyzed to identify one or more source determination outcomes (120). The one or more source determination outcomes may be included in internal or external reports, which may be reported as a physical report, or displayed in a user interface. For instance, a user interface may display the one or more source determination outcomes. The user interface may also allow a user to navigate and refine the outcomes (e.g., select a specific a particular set of outcomes), or compare one or more source determination outcomes corresponding to different food products, different lots of the same food products, the same product lot at different lots in the food production chain, or any combination thereof. At 122, the one or more allergen or toxigen presence outcomes, or the one or more source determination outcomes, may results in business-level decisions, such as implementing changes in supplier management, in factory process management, or in quality control processes.

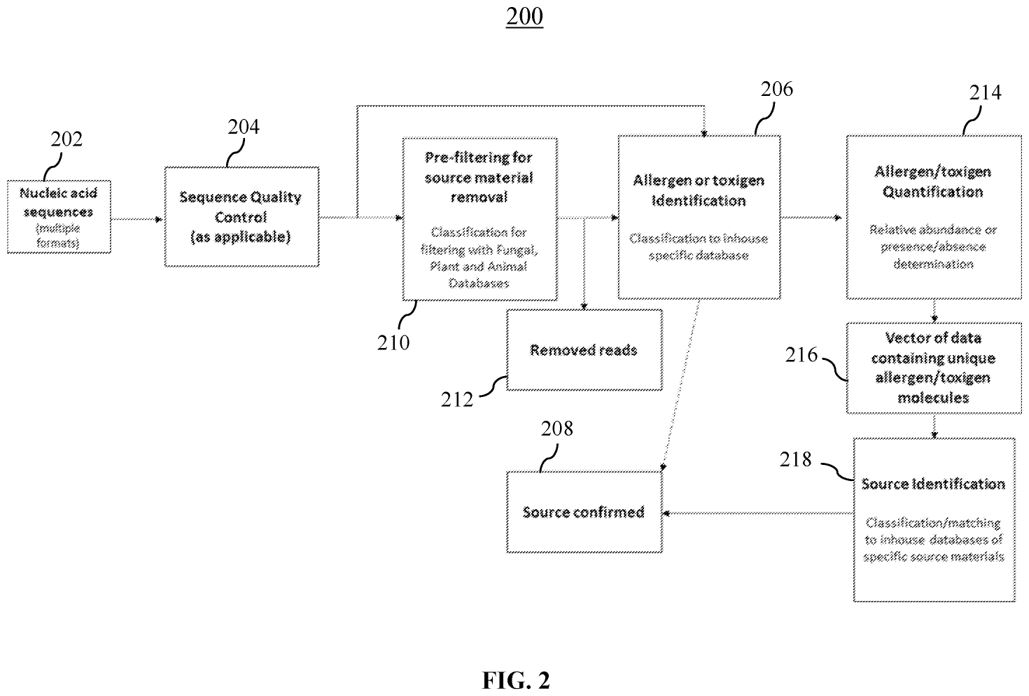

FIG. 2 depicts an exemplary data analysis process for identifying one or more allergen or toxigen sequences corresponding to one or more allergens or toxigens present in a food product (method 200). The one or more allergen or toxigen sequences may correspond to all allergens or toxigens present in the food sample, or to a partial list. For example, one or more allergen or toxigen sequences may correspond only to allergens or toxigens present in the food product over a pre-determined threshold value. Common allergens that can be present in food include, without limitation, milk, peanut or groundnut, egg and shellfish. The toxigen may be a toxigen produced by a microbe, a fungi or a plant.

In an exemplary method for allergen or toxigen detection, nucleic acid sequences are received at 202. The nucleic acid sequences may correspond to DNA, RNA, or both DNA and RNA sequences. The nucleic acid sequences may be provided in any suitable format. At 204, a sequence quality control may be implemented as applicable. The sequence quality control may include, without limitation, trimming, length filtering, sequencing adapter removal, sequence binning, or any combination thereof. The nucleic acid sequences are then analyzed for allergen or toxigen identification (206), in which one or more sequences corresponding to one or more antigens or toxigens are identified. Microbial identification may include classification to an allergen or toxigen database (e.g. an in-house allergen or toxigen database). The allergen or toxigen databases may be specific to a particular category of allergens or toxigens or may correspond to a wide range of allergens or toxigens. The allergen or toxigen databases may also correspond to allergens or toxigens commonly detected in a particular food source or set of food sources, or to allergens or toxigens of particular concern. Any suitable database corresponding to allergen or toxigen sequences may be used for allergen or toxigen identification. The databases may correspond to nucleic acid sequences consisting of combinations of nucleotides (e.g., A, T, G, or C), or they may correspond to amino acid sequences corresponding to one or more allergen or toxigen proteins or protein isoforms encoded by said nucleic acids, and which may include any naturally and non-naturally occurring amino acid residues known in the art. After allergen or toxigen identification, a source for the antigen or toxigen can be confirmed (208). Prior to allergen or toxigen identification, a pre-filtering step may be performed for removal of sequences corresponding to the source material (210). The pre-filtering step can include classification of sequences using fungal, plant, or animal databases. In some instances, pre-filtering can identify sequence reads that do not correspond to any allergen or toxigen present in the food product (e.g. unmapped sequences). Pre-filtering can also be used to identify sequence reads corresponding to the food raw materials. At 212, the pre-filtered sequence reads may then be removed from the sequence data.

Allergen or toxigen quantification (214) may also be performed. The allergen or toxigen quantification may be determined as the relative abundance of one or more allergen or toxigen, or as a presence or absence determination. Allergen or toxigen quantification may be based on the number of reads and/or the coverage of reads corresponding to a particular allergen or toxigen. For example, a higher read count for sequences corresponding to a particular allergen or toxigen would indicate higher levels of that microbe in the food product. The quantification may be based on an internal or external control sample. In some instances, allergen or toxigen quantification may include setting a threshold. For example, the presence or absence of a microbe may be determined based on whether sequence reads corresponding to that sample surpass a pre-determined threshold, such as, at least 90%, at least 95%, or a 100% sequence identity match to at least one sequence in a reference database. In other cases, the presence or absence of a microbe may be determined based on whether the number of sequence reads corresponding to a particular allergen or toxigen surpass a pre-determined threshold, such as having at least 10 unique reads. Following microbial quantification, a vector data containing unique allergen or toxigens is generated (216). The vector data is used for secondary source identification at 218. Secondary source identification may include, for example, classification or matching of the allergen or toxigen sequences to one or more allergen or toxigen databases. For example, in-house allergen or toxigen databases corresponding to allergens or toxigens associated with specific source materials may be used for secondary source identification. Allergen or toxigen sources identified by secondary source identification are then confirmed at 208.

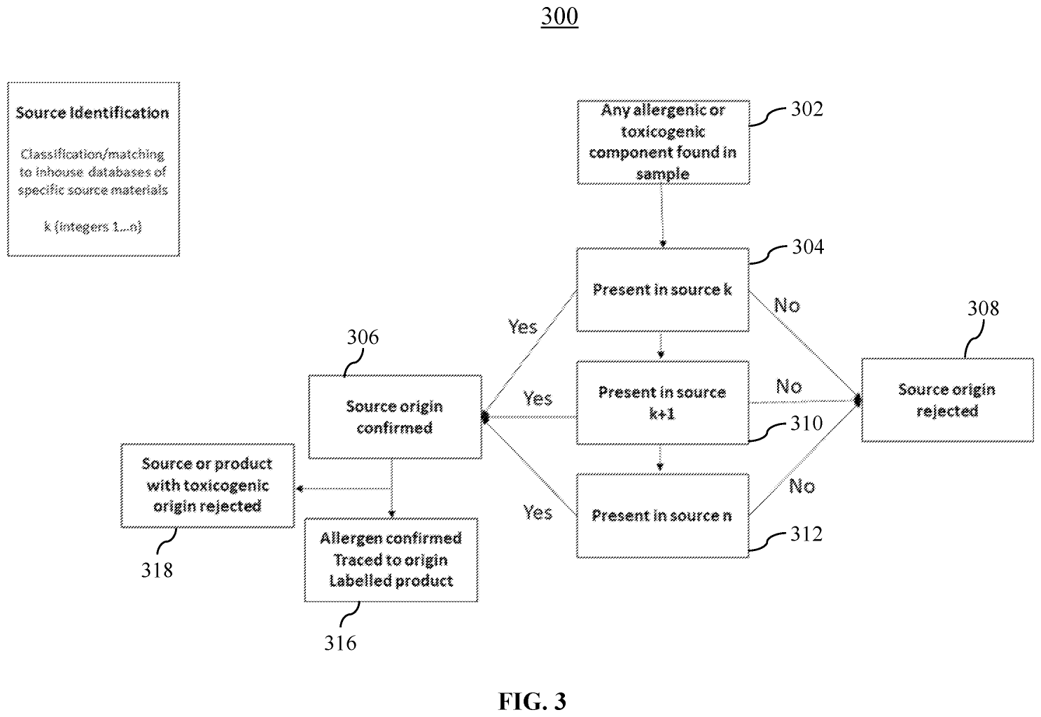

FIG. 3 shows an exemplary process of determining whether antigen or toxigen sequences in the sequence data\correspond to a particular antigen or toxigen detection of an allergen or toxigen in a food sample (method 300). The method corresponds to allergen or toxigen classification and may involve classification or matching of allergen or toxigen sequences to in-house allergen or toxigen databases for specific source materials k (integers k+1 . . . n). The determination is performed as an iterative process, where only one food source is considered at each stage or step of the process. Allergen or toxigens sequences found in the sample is received at 302. At 304, the allergen or toxigen sequences are analyzed to determine whether the allergen or toxigen is present in source k. If the allergen or toxigen is present in food source k, the source is confirmed as a source of the allergen or toxigen at 306. Alternatively, if the allergen or toxigen is not present in source k, the source is rejected as the source of the allergen (308). At 310, the analysis is iterated to determine whether the allergen or toxigen is present in source k+1. The source is confirmed (306) if the allergen or toxigen is present in source k+1 but rejected (308) if the allergen or toxigen is not present in source k+1. The analysis is further iterated for source n (312) with the source confirmed (306) if the allergen or toxigen is present in food source n, but rejected (308) if the allergen or toxigen is not present in source n. Sources confirmed at step 306 are then used for confirmation of the source of the allergen, trace-back of the allergen to its origin (e.g., a particular supplier or raw material), and/or for product labelling purposes (316). Sources or products confirmed to contain a toxigen are rejected at 318.

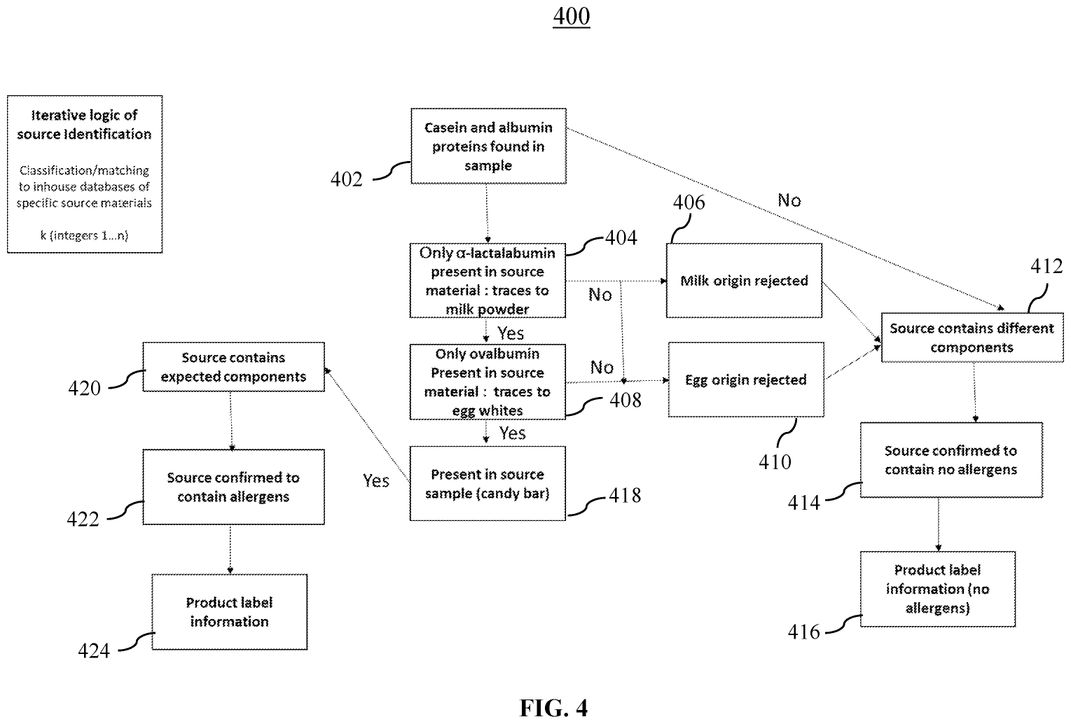

FIG. 4 depicts an example of allergen detection in a food sample (method 400). Allergen detection may be performed by classification or matching to in-house allergen databases of specific allergenic source materials. The determination is displayed as an iterative process, where one allergenic source can be considered for presence/absence at each stage or step of the process. In this example, nucleic acid sequences corresponding to casein and albumin proteins are found in a sample from a particular food product (402). At 404, only α-lactalbumin is detected in the source material of the food sample. Milk powder is rejected as an origin at step 406 based on the determination that α-lactalbumin was not traced to milk powder. Subsequently, at 408, ovalbumin is detected in the source material of the food sample. Egg is rejected as an origin at step 410 based on the determination that ovalbumin was not traced to egg whites, another component of the food sample. At 412, the determination is made that the food source contains different components than an allergenic source, and at 414, the food source is confirmed to contain no allergens. The product label information is prepared to indicate that no allergens were found in the food product (416). At 418, the casein and albumin sequences are determined to be present in a particular source (a candy bar). The determination that the food source contains expected allergenic components of the food product is then made (420), and the source is confirmed to contain allergens at 422. The product label information is then updated accordingly at 424, to reflect the presence of allergens in the food sample. A method similar to method 400 may be used for detection of toxigens in a food sample.

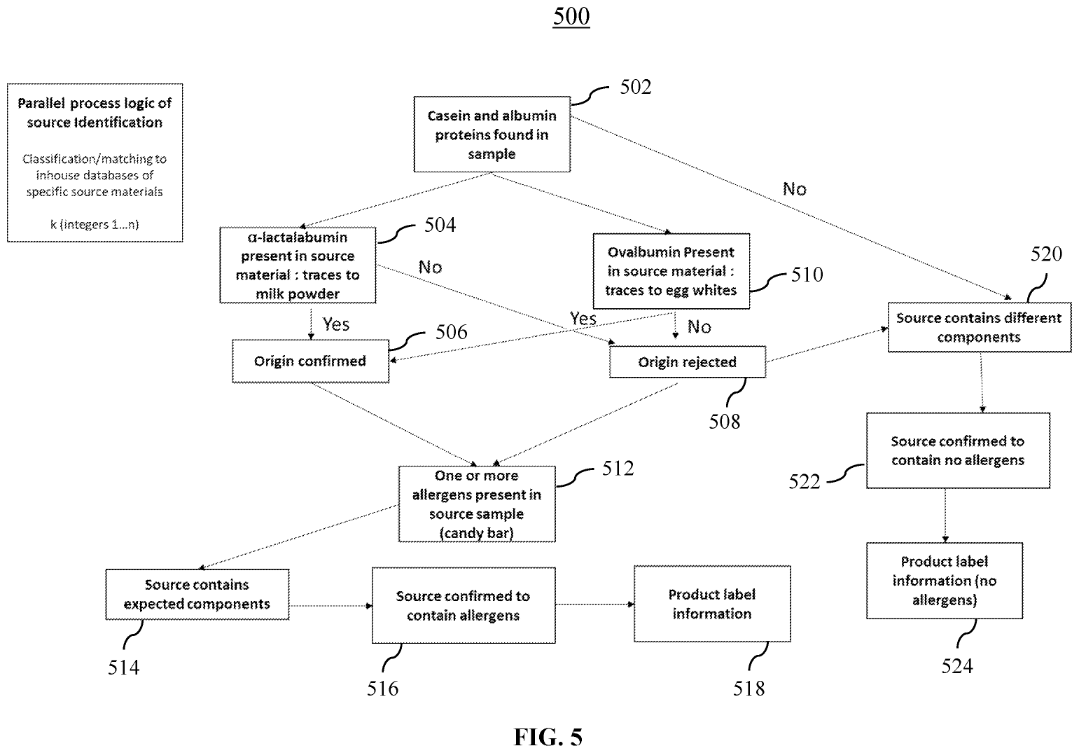

Allergen or toxigen determination may also be performed as a parallel process. FIG. 5 depicts an example of allergen detection in a food sample (method 500). Allergen detection may be performed by classification or matching to in-house allergen databases of specific allergenic source materials. In this method, the allergenic determination is displayed as a parallel process, where all allergenic sources can be jointly considered for presence/absence in parallel steps. In this example, nucleic acid sequences corresponding to casein and albumin proteins are found in a sample from a particular food product (502). At 504, α-lactalbumin is detected in the source material of the food sample, and milk powder is confirmed at step 506 based on the determination that α-lactalbumin traces to milk powder. Alternatively, if no α-lactalbumin is detected in the source material of the food sample at 504, milk powder is rejected as an origin (506). In parallel to step 504, ovalbumin is detected in the source material of the food sample at 501. Egg is confirmed as an origin at step 506 based on the determination that ovalbumin traces to egg whites. If no ovalbumin is detected in the source material at 510, egg powder is instead rejected as an origin (508). At 512, the one or more allergens determined to be present in a particular source (a candy bar). The determination that the food source contains expected allergenic components of the food product is then made (514), and the source is confirmed to contain allergens at 4516. The product label information is then updated accordingly at 514, to reflect the presence of allergens in the food sample. In the case that casein and albumin are not detected at 502, or that all examined origins are rejected at 508, the determination is made that the food source contains different components than an allergenic source (520). Consequently, at 522 the food source is confirmed to contain no allergens, and the product label information is prepared to indicate that no allergens were found in the food product (524). A method similar to method 500 may be used for detection of toxigens in a food sample.

A particular allergen or toxigen may also be traced back to a particular supplier. For example, an allergen detected in the food product during food production may be detected and traced back to a particular supplier. After analysis of sequence data corresponding to a food product, a particular allergen (e.g., a shellfish or egg allergen) may be identified during one point in the food production chain. The allergen can then be matched to raw materials provided by a particular supplier for that step of the food production chain. The food producer may consider those raw materials as compromised and may then decide to implement a corrective action, such as issuing a warning to the supplier or changing to a different supplier altogether. In this way, the methods disclosed herein may be used for end-to-end trace-back of allergens or toxigens during the food production process. By evaluating a food product at one or more steps of the food production chain, the producer can make sure that no allergens or toxigens are detected and that allergenic or toxigenic raw materials are removed from the food production process.

The methods for detecting an antigen or a toxigen in a food sample disclosed herein may be combined with other nucleic acid sequence-based analysis methods. For example, the sequence data obtained by the method for detecting the presence of an allergen or toxigen may be analyzed to identify one or more microbial signatures. The one or more microbial signatures may correspond to one or more microbes present in the food product. The one or more microbial signatures may then be used to authenticate or identify a source for the food product in addition to detecting the presence of an allergen or toxigen. The microbial signatures may be identified in in parallel or subsequent to identification of allergen and toxigen sequences in the food sample.

### II. Nucleic Acid Sequence Data

The methods disclosed herein comprise obtaining sequence data for a plurality of nucleic acid sequences present in a food product. The sequence data may correspond to any nucleic acid present in a food sample. For instance, the sequence data may correspond to a plurality of DNA and/or RNA sequences. The plurality of nucleic acid sequences may correspond to nucleic acids from one or more allergens or toxigens present in food sample, or to nucleic acids from the food product.

Obtaining the sequence data can include extracting nucleic acids from the food product. Methods of extracting nucleic acids known in the art may be used. Without being limited, nucleic acids may be extracted using TrizolLS reagent, phenol: chloroform: isoamyl alcohol extraction, or equivalents. Nucleic acid extraction may also be performed using commercially available kits, such as, Ambion RNA isolation kits (e.g., Purelink RNA Mini kit or DynaBeads mRNA direct micro kit), MAgmax FFPE total nucleic acid isolation kit, Pall DNA and RNA Purification kits, Qiagen Allprep, PowerViral, Powersoil, or PowerMag kits, NEBNext Microbiome DNA Enrichment kit, or equivalents. Nucleic acid extraction may be performed using frozen or fresh samples. For example, a food product may be fixed before nucleic acid extraction. Nucleic acid extraction may also include a step of cell lysis. Cell lysis may be performed through any methods known to those skilled in the art, including, but not limited to, enzymatic lysis using lytic enzymes such as lysozyme, lysostaphin, mutanolysin, proteinase K, subtilisin, or any combination thereof; physical shearing, such as with glass beads, sonication, ultrasound, or high pressure; and any other cell lysis method known to those skilled in the art.

It should be understood that the present teachings contemplate sequence data that may be obtained using all available varieties of techniques, platforms, or technologies, including, but not limited to: capillary electrophoresis, microarrays, ligation-based systems, polymerase-based systems, hybridization-based systems, in situ sequencing, direct or indirect nucleotide identification systems, pyrosequencing, ion- or pH-based detection systems, electronic signature-based systems, etc.

The sequence data may be obtained by any method available in the art, such as by nucleic acid sequencing (e.g., next generation sequencing) or microarray analysis. The methods disclosed herein are not dependent upon a particular next generation sequencing technology, and the user needs to make appropriate choices for the intended downstream sequencing platform according to manufacturers' protocols. Exemplary sequencing platforms that may be used to obtain sequence data according to the methods disclosed herein include, but are not limited to, those produced by Illumina®, Oxford Nanopore™, Ion Torrent™, Roche™, Pacific Biosciences™, and Life Technologies™.

Depending on the sequencing technology used with the methods, a sequencing library may be prepared. The sequencing library will be representative of nucleic acids present in a food product and can be used with next generation sequencing platforms. Sequencing library preparation can include nucleic acid fragmentation, sample indexing, adaptor ligation, and library normalization. Sample indexing or barcoding allows multiple samples to be run simultaneously, taking full advantage of the high-throughput nature of current sequencing platforms. Adapter ligation is sequencing platform specific and standard to manufacturers' protocols. The adaptors may contain sequencing platform-specific end sequences and index sequences that allow for de-convolution of sequence data by sample. Barcoding and adapter ligation may be performed by any method known to those in the art and may be adapted for analysis of the sequencing library with a particular sequencing platform. Library preparation can also include amplification, concentration, or dilution of the sequencing library. Libraries can be prepared at platform-specific concentrations of DNA and typically require amplification, concentration, or dilution to achieve the required concentration. The concentration of nucleic acids in the sequencing library may be determined by quantitative real-time PCR using platform specific manufacturer protocols or fluorescence-based measurement known in the art. In some instances, preparing the sequencing library includes selective enrichment of specific target nucleic acids or regions.

Nucleotide sequences of individual molecules are determined in a platform-specific manner to produce a raw dataset. The raw dataset can be converted to nucleotide sequencing information corresponding to each molecule in a sequencing library. The resulting products are whole “reads,” which may be processed to determine information about the food product. The sequence data may be produced in any format, such as BAM files, which are sequencing platform-independent and ready for bioinformatics analysis. Additional file types may include FASTA and FASTQ file formats, or other manufacturer-specific formats that can be converted to BAM, VCF, FASTQ, or FASTA format.

Once obtained, sequence data may be transferred in real time from the instrument used to generate sequence data as soon as the sequence data has reached a sufficient size in total base pairs for analysis, or it may be stored in a database until further analysis.

The sequence data may then be prepared for further analysis. This preparation can include performing sequence quality control, trimming, length filtering, sequencing adapter removal, and/or binning of reads by molecular barcode from the sequencing reads. In particular, the reads that represent the plurality of nucleic acid sequences from a food product can be quality controlled to remove the adapter sequences, clonal reads due to PCR amplification, and platform-specific sequence errors and filtered to achieve an acceptable error rate. Sequencing reads in the sequencing data may be deconstructed into, for example, k-mers of a particular size. Exemplary k-mer based methods that may be used include, without limitation, Kraken (Wood, et al. (2019), Genome biology, 20(1): 257; Wood, and Salzberg (2014) Genome biology, 15(3): R46), Basic Local Alignment Search Tool (BLAST), Mash (Ondov, et al. (2016), Genome biology, 17(1): 132) or MUMmer (Kurtz, et al. (2004) Genome biology, 5(2): R12), or any equivalent analysis platform available to those skilled in the art. Sequence assembly, mapping, or pairwise comparison of the sequencing reads in the sequence data may also be performed. In some cases, nucleic acid sequences corresponding to the food product or another agent can be filtered or removed from the sequence data prior to further analysis. In some embodiments, the sequence data may correspond to nucleic acid sequences encoding an allergen or toxigen present in the food sample. The nucleic acid sequences may be translated in silico prior to analysis of the sequence data.

In some instances, sequence data from various food samples may be collated and used for further analysis. Additional statistical analysis may be applied to the collated sequence data to determine large scale patterns. Statistical analysis methods that can be used to assess large-scale patterns in sequence data include, but are not limited to, statistical probability, regression, analysis of variance and statistical significance, principal component analysis, multivariate regression, multivariate analyses of variance, time series analysis models, and statistical bootstrapping. More advanced analytical techniques such as hidden Markov models, Markov Chain Monte Carlo sampling, and machine learning algorithms such as linear regression, support vector machines, random forests, or machine learning algorithms classified under supervised learning, unsupervised learning, and reinforcement learning methods, may be applied for large scale pattern detection and outcome determination at various analytical scales.

### III. Allergens and Toxigens

The methods described herein can include identifying one or more allergen or toxigen sequences in the sequence data. The one or more allergen or toxigen sequences may correspond to one or more allergens or toxigens present in a food product may correspond to one or more microbes present in the food product.

Identifying the one or more allergen or toxigen sequences can include comparing sequence data to one or more databases. The databases can contain sequences (e.g., nucleic acid sequences, or amino acid sequences) from a particular group of allergens or toxigens. For instance, the databases may correspond to nucleic acid sequences from allergens associated with particular allergenic sources. Any publicly available database that is suitable for allergen or toxigen identification may be used. Alternatively, an in-house database may be generated and used to identify allergen or toxigen sequences.

The identified allergen or toxigen sequences may be used to detect the present of an allergen or toxigen in a food sample. Detection of the allergen or toxigen may be based on whether the allergen or toxigen sequences are above a predetermined threshold. The pre-determined threshold may correspond to the relative level or sequence coverage of a sequence in the food sample. The relative level of the sequence can be indicative of the relative abundance of the allergen or toxigen in the food product. The threshold may be set in terms of, for example, a Ct value, a nucleic acid copy number, a minimum number of sequencing reads, a concentration (e.g., in mg/mL or mg/L units), etc.

The allergens may be of any origin, including plant or animal origin. Examples of allergens which may be present in food include, without limitation, eggs, milk, meat, fishes, crustacea and mollusks, cereals, legumes and nuts, fruits, vegetables, beer yeast, and gelatin. More particularly, egg white and egg yolk of the eggs, milk and cheese of the milk, pork, beef, chicken and mutton of the meat, mackerel, horse mackerel, sardine, tuna, salmon, codfish, flatfish and salmon caviar of the fishes, crab, shrimp, blue mussel, squid, octopus, lobster and abalone of the crustacea and mollusks, wheat, rice, buckwheat, rye, barley, oat, corn, millet, foxtail millet and barnyard grass of the cereals, soybean, peanut, cacao, pea, kidney bean, hazelnut, Brazil nut, almond, coconut and walnut of the legumes and nuts, apple, banana, orange, peach, kiwi, strawberry, melon, avocado, grapefruit, mango, pear, sesame and mustard of the fruits, tomato, carrot, potato, spinach, onion, garlic, bamboo shoot, pumpkin, sweet potato, celery, parsley, yam and Matsutake mushroom, or the foods containing any of the allergens and the ingredients thereof (e.g., ovoalbumin, ovomucoid, lysozyme, casein, beta-lactoglobulin, alpha-lactoalbumin, gluten, and alpha-amylase inhibitor). In some instances, the allergen is a milk, egg or shellfish allergen.

The toxigen may be, for example, of bacterial, fungal or plant origin. Toxigens which may be detected with the present methods include, but are not limited to, staphylococcal toxins, enterotoxins (e.g., enterotoxin B), streptococcal toxins, shiga toxins, botulinum toxin, aflatoxins, and ricin.

### IV. Systems

In one aspect, the disclosure provides systems for performing any of the methods of the disclosure.

The system can be configured to detect an allergen in a food product. For example, the system may include one or more processors and a memory comprising instructions executable by the one or more processors. When executed by the one or more processors, the instructions may cause the system to obtain sequence data for nucleic acid sequences present in a food product; identify one or more allergen sequences in the sequence data, wherein the one or more allergen sequences correspond to an allergen present in the food sample; and detect an allergen in the food product. The system may also be configured to detect the presence of the allergen in the food product if the one or more allergen sequences are above a predetermined threshold. The allergen may be any allergen described herein, such as an allergen or milk or egg origin.

The system can also be configured to detect a toxigen in a food product. For example, the system may include one or more processors and a memory comprising instructions executable by the one or more processors. When executed by the one or more processors, the instructions may cause the system to obtain sequence data for nucleic acid sequences present in a food product; identify one or more toxigen sequences in the sequence data, wherein the one or more toxigen sequences correspond to a toxigen present in the food sample; and detect a toxigen in the food product. The system may also be configured to detect the presence of the allergen in the food product if the one or more toxigen sequences are above a predetermined threshold. The toxigen may be any toxigen described herein, such as a toxigen produced by a bacteria, fungi or plant.

The systems may be configured to to detect the presence of the allergen in the food product if the one or more toxigen sequences are above a predetermined threshold corresponding to the relative level of a sequence in the food sample. Alternatively, the predetermined threshold may correspond to the sequence coverage of a sequence in the food sample.

The sequence data obtain by the system may correspond to any of the allergens and may include preparing a sequencing library. For instance, the sequence data may be obtained from next generation sequencing, or microarray analysis, and may correspond to DNA or RNA sequences (e.g., mRNA sequences encoding allergens or toxigens present in a food sample). In some instances, the system may be configured further to convert RNA sequences into amino acid sequences corresponding to polypeptides encoded by the RNA sequences prior to identifying the one or more allergen sequences or the one or more toxigen sequences.

In some instances, the sequences corresponding to the food product may be filtered from the sequence data prior to identifying the one or more allergen sequences or the one or more toxigen sequences. Sequences corresponding to microbes present in the product may also be filtered from the sequence data prior to identifying the one or more allergen sequences or the one or more toxigen sequences. The system may be configured to identify the one or more allergen or toxigen sequences by comparing the sequence data against one or more databases of allergen or toxigen sequences. The databases may include any suitable allergen or toxigen database, such as any database described herein.

The systems may also be configured to detect an allergen or a toxigen at two or more points in a food production chain of the food product. Moreover, the systems may be also be configured to trace an allergen or toxigen to a particular supplier of the food product.

### V. Methods in Computer-Readable Storage Devices

Any of the methods described herein can be implemented by computer-executable instructions or code stored in one or more computer-readable medium (e.g., a memory, a magnetic storage, an optical storage, or the like). Such instructions can cause one or more processors to implement the method.

### EXAMPLES

### Example 1

This example describes the use of metagenomics filtering to detect the presence of allergens or toxigens in a food product.

### Materials and Methods

### Sample Collection, Preparation, and Sequencing

Milk powder, animal meal, corn meal, or egg powder samples were collected from a local market in the United States. Sample preparation, total RNA extraction and integrity confirmation, cDNA construction, and library preparation for these samples was previously described by Haiminen, N., et al. (2019) NPJ Sci Food 3:24.

The samples were used to extract total RNA as described by Chen, P., et al. (2017) Pathogens 6:68, and total DNA as described elsewhere (Weis, A. M., et al. (2016). Appl. Environ. Microbiol 82:7165-7175; Emond-Rheault, J.-G., et al. (2017) Front. Microbiol. 8:996; Miller, B., et al. (2015) Kapa Biosyst. Appl. Note 1-8 (2015); Lüdeke, C. H. M., et al. (2015) Genome Announc. 3:2-3; Jeannotte, R., et al. (2015) Agil. Appl. Note 1-8; Arabyan, N., et al. (2016) Sci Rep 6:29525). DNA and RNA purity (A_{260/230} and A_{260/280} ratios ≥1.8) and integrity were confirmed with Nanodrop (Nanodrop Technologies, Wilmington, DE, USA) and BioAnalyzer RNA Kit (Agilent Technologies Inc., Santa Clara, CA, USA) (Chen, P., et al. (2017) Pathogens 6:68). For RNA samples, cDNA was prepared using 4 to 15 μg total input of RNA and the SuperScript Double Stranded cDNA Synthesis kit (Invitrogen, Catalog no. 11917-020, Life Technology Carlsbad, CA).

Sequencing libraries were prepared using HyperPrep Plus (Kapa BioSystems, Wilmington, MA, USA) as previously described (Chen, P., et al. (2017) Pathogens 6:68; Chen, P., et al. (2017) Appl Env. Microbiol 83; Kol, A., et al. (2014) Stem Cells Dev 23:1831-1843), with an insert size between 300-400 bp. Library quantification was performed using qPCR (Library Quantification kit, catalog no. KK4824, Illumina, San Diego, CA) prior to submission for sequencing. The Illumina HiSeq 4000 (San Diego, CA) was used with 150 paired-end chemistry for each sample except the following: HiSeq 2000 with 100 paired-end chemistry was used for the four preliminary samples, and HiSeq 3000 with 150 paired-end chemistry was used for two other samples (MFMB-04 and MFMB-17).

### Sequence Data Quality Control

Illumina Universal adapters were removed, and reads were trimmed using Trim-Galore (Morgulis, A., et al. (2006) J. Comput. Biol. 13:1028-1040) with a minimum read length parameter 50 bp. The resulting reads were filtered using Kraken software as described below with a custom database built from the PhiX genome (NCBI Reference Sequence: NC_001422.1). Trimmed non-PhiX reads were used in subsequent matrix filtering and microbial identification steps.

### Matrix Filtering Process and Validation

Kraken (Wood, D. E., and Salzberg, S. L. (2014) Genome Biol. 15: R46), with a k-mer size of 31 bp, was used to identify and remove reads that matched a pre-determined list of 31 common food matrix and potential contaminant eukaryotic genomes. These food matrix organisms were chosen based on preliminary eukaryotic read alignment experiments of the samples as well as high-volume food components in the supply chain. Because of the large size of eukaryotic genomes in the custom Kraken database, a random k-mer reduction was applied to reduce the size of the database by 58% (using Kraken-build with option “—max-db-size”), in order to fit the database in 188 GB for in-memory processing. A conservative Kraken score threshold of 0.1 was applied to avoid filtering microbial reads. The matrix-filtering database includes low complexity and repeat regions of eukaryotic genomes to capture all possible matrix reads. This filtering database and the score thresholds were also used in the matrix filtering for in silico testing as described below.

### Allergen and Toxigen Identification.

Remaining reads after quality control and matrix filtering were classified using Diamond software v2.0.11 against a database of allergen or toxigens using the BLASTx search option settings for each sample. The BLASTx option performs a rapid translated search of nucleic acid sequences against protein databases. A minimum of 10 reads matching for both forward and reverse paired data was required as the threshold for positive presence determination at 90% identity. Prior to initiating the search, a Diamond software-compatible database of protein sequences of known allergens and their isoforms, or of toxins or toxigenic proteins, was prepared.

### Results

Milk powder samples were collected from a food manufacturing line and analyzed for the presence of allergens and toxigens as a proof-of-principle of the method depicted in FIGS. 1-5.

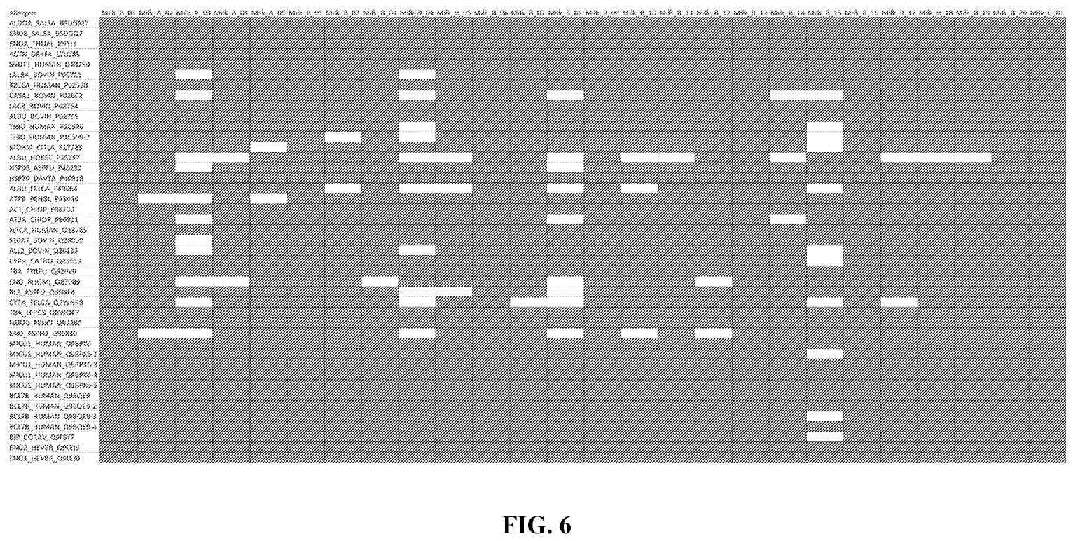

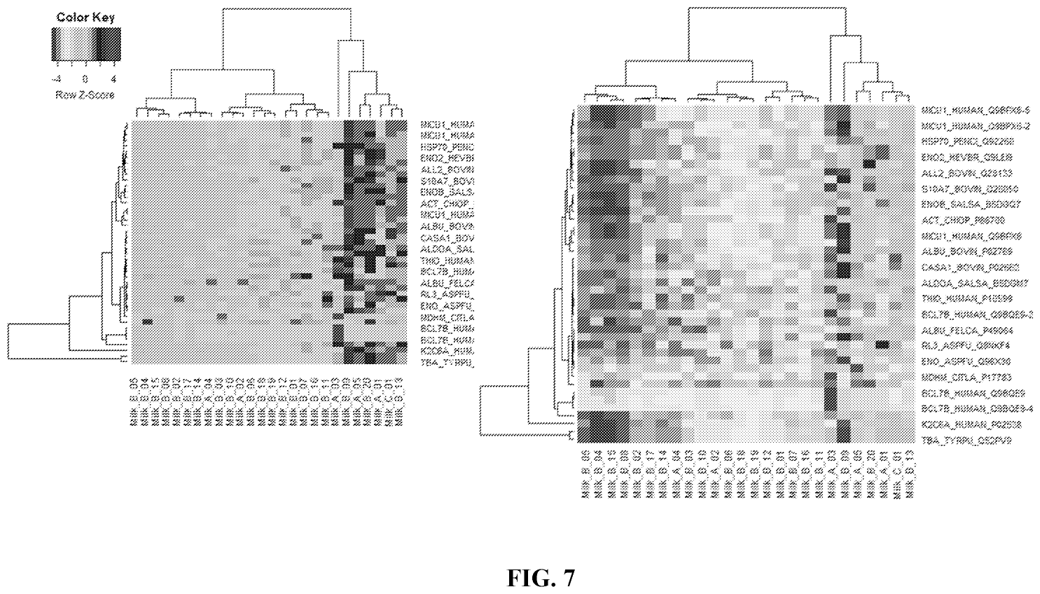

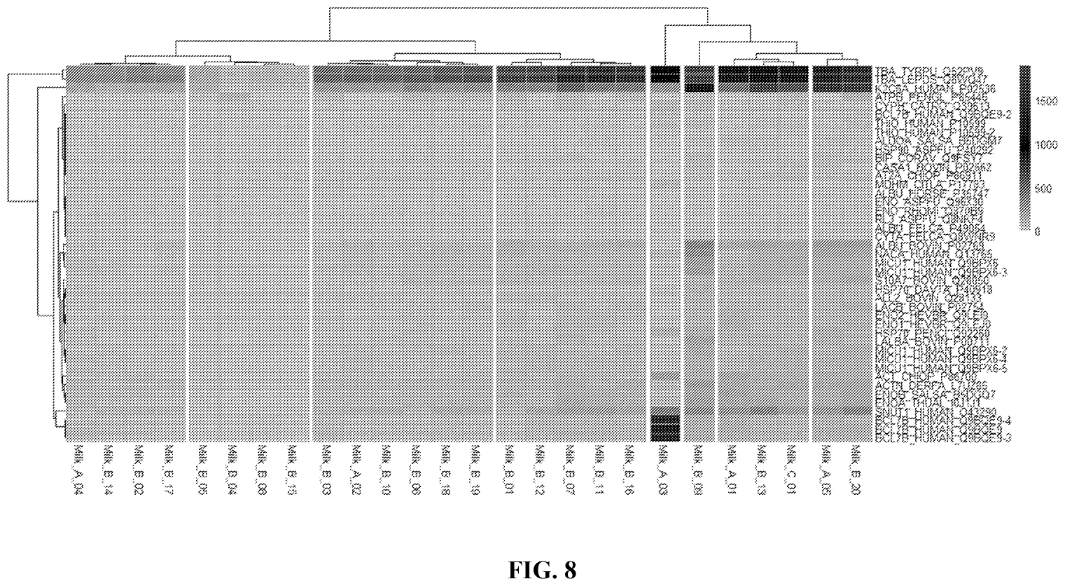

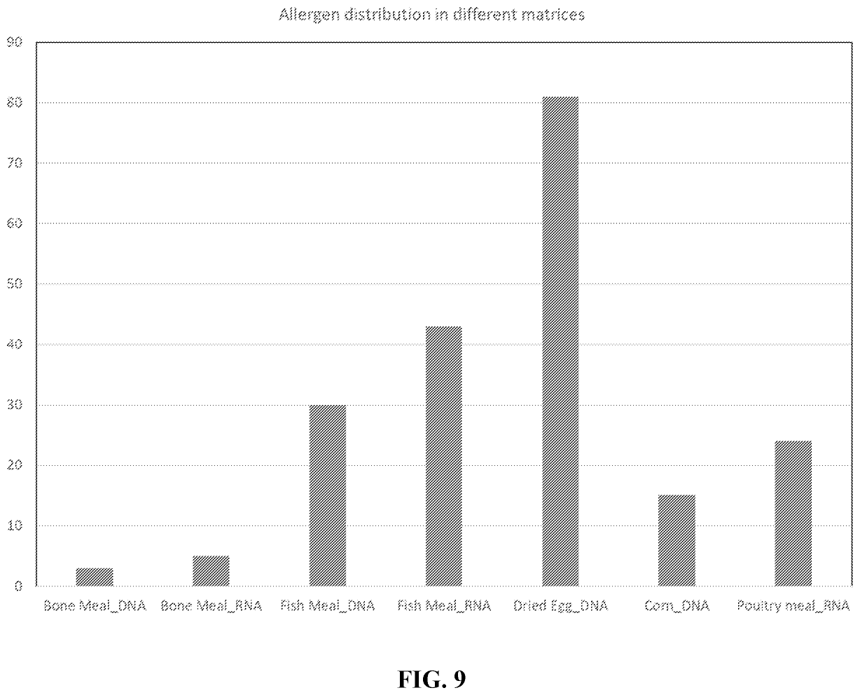

Table 1 summarizes the allergens identified in the milk powder samples, with the sources of the allergens identified at the genus taxonomic level. FIG. 6 depicts a heatmap for the presence or absence of the different allergens included in the analytical database used for allergen detection, while FIGS. 7-8 depict the relative level of the allergens found in the milk powder samples. FIG. 9 depicts the distribution of allergens across the different matrices tested. In addition to allergens, known components of milk powder were also identified in the analysis (Table 1, bolded), validating the detection process.

TABLE 1

| Database Name | |
|---|---|
| of allergen | Description |
| ALDOA_SALSA | Fructose-bisphosphate aldolase A OS = Salmo salar OX = 8030 PE = 1 SV = 1 |
| ENOB_SALSA | Beta-enolase OS = Salmo salar OX = 8030 GN = ENO3 PE = 1 SV = 1 |
| ENOA_THUAL | Alpha-enolase OS = Thunnus albacares OX = 8236 GN = ENO1 PE = 1 SV = 1 |
| ACTN_DERFA | Alpha-actinin OS = Dermatophagoides farinae OX = 6954 PE = 1 SV = 1 |
| SNUT1_HUMAN | U4/U6.U5 tri-snRNP-associated protein 1 OS = Homo sapiens OX = 9606 |
| | GN = SART1 PE = 1 SV = 1 |
| LALBA_{—}BOVIN | Alpha-lactalbumin OS = Bos taurus OX = 9913 GN = LALBA PE = 1 |
| | SV = 2 |
| K2C6A_HUMAN | Keratin, type II cytoskeletal 6A OS = Homo sapiens OX = 9606 |
| | GN = KRT6A PE = 1 SV = 3 |
| CASA1_{—}BOVIN | Alpha-S1-casein OS = Bos taurus OX = 9913 GN = CSN1S1 PE = 1 SV = 2 |
| LACB_{—}BOVIN | Beta-lactoglobulin OS = Bos taurus OX = 9913 GN = LGB PE = 1 SV = 3 |
| ALBU_{—}BOVIN | Albumin OS = Bos taurus OX = 9913 GN = ALB PE = 1 SV = 4 |
| THIO_HUMAN | Thioredoxin OS = Homo sapiens OX = 9606 GN = TXN PE = 1 SV = 3 |
| THIO_HUMAN | Isoform 2 of Thioredoxin OS = Homo sapiens OX = 9606 GN = TXN |
| MDHM_CITLA | Malate dehydrogenase, mitochondrial OS = Citrullus lanatus OX = 3654 |
| | GN = MMDH PE = 1 SV = 1 |
| ALBU_HORSE | Albumin OS = Equus caballus OX = 9796 GN = ALB PE = 1 SV = 1 |
| HSP90_ASPFU | Heat shock protein 90 OS = Neosartorya fumigata (strain ATCC MYA- |
| | 4609 / Af293 / CBS 101355 / FGSC A1100) OX = 330879 GN = hsp90 |
| | PE = 1 SV = 3 |
| HSP70_DAVTA | Heat shock 70 kDa protein OS = Davidiella tassiana OX = 29918 |
| | GN = HSP70 PE = 1 SV = 1 |
| ALBU_FELCA | Albumin OS = Felis catus OX = 9685 GN = ALB PE = 1 SV = 1 |
| ATPB_PENGL | ATP synthase subunit beta, mitochondrial (Fragments) OS = Penicillium |
| | glabrum OX = 69773 GN = atp2 PE = 1 SV = 2 |
| ACT_CHIOP | Actin, muscle (Fragments) OS = Chionoecetes opilio OX = 41210 PE = 1 |
| | SV = 1 |
| AT2A_CHIOP | Sarcoplasmic/endoplasmic reticulum calcium ATPase (Fragments) |
| | OS = Chionoecetes opilio OX = 41210 PE = 1 SV = 1 |
| NACA_HUMAN | Nascent polypeptide-associated complex subunit alpha OS = Homo sapiens |
| | OX = 9606 GN = NACA PE = 1 SV = 1 |
| S10A7_{—}BOVIN | Protein S100-A7 OS = Bos taurus OX = 9913 GN = S100A7 PE = 1 SV = 1 |
| ALL2_{—}BOVIN | Allergen Bos d 2 OS = Bos taurus OX = 9913 PE = 1 SV = 1 |
| CYPH_CATRO | Peptidyl-prolyl cis-trans isomerase OS = Catharanthus roseus OX = 4058 |
| | GN = PCKR1 PE = 1 SV = 1 |
| TBA_TYRPU | Tubulin alpha chain OS = Tyrophagus putrescentiae OX = 59818 PE = 1 |
| | SV = 1 |
| ENO_RHOMI | Enolase OS = Rhodotorula mucilaginosa OX = 5537 GN = ENO PE = 1 SV = 1 |
| RL3_ASPFU | 60S ribosomal protein L3 OS = Neosartorya fumigata (strain ATCC MYA- |
| | 4609 / Af293 / CBS 101355 / FGSC A1100) OX = 330879 GN = rpl3 PE = 1 |
| | SV = 2 |
| CYTA_FELCA | Cystatin-A OS = Felis catus OX = 9685 GN = CSTA PE = 1 SV = 1 |
| TBA_LEPDS | Tubulin alpha chain OS = Lepidoglyphus destructor OX = 36936 PE = 1 |
| | SV = 2 |
| HSP70_PENCI | Heat shock 70 kDa protein (Fragment) OS = Penicillium citrinum |
| | OX = 5077 GN = HSP70 PE = 1 SV = 1 |
| ENO_ASPFU | Enolase OS = Neosartorya fumigata (strain ATCC MYA-4609 / Af293 / |
| | CBS 101355 / FGSC A1100) OX = 330879 GN = enoA PE = 1 SV = 3 |
| MICU1_HUMAN | Calcium uptake protein 1, mitochondrial OS = Homo sapiens OX = 9606 |
| | GN = MICU1 PE = 1 SV = 1 |
| MICU1_HUMAN | Isoform 2 of Calcium uptake protein 1, mitochondrial OS = Homo sapiens |
| | OX = 9606 GN = MICU1 |
| MICU1_HUMAN | Isoform 3 of Calcium uptake protein 1, mitochondrial OS = Homo sapiens |
| | OX = 9606 GN = MICU1 |
| MICU1_HUMAN | Isoform 4 of Calcium uptake protein 1, mitochondrial OS = Homo sapiens |
| | OX = 9606 GN = MICU1 |
| MICU1_HUMAN | Isoform 5 of Calcium uptake protein 1, mitochondrial OS = Homo sapiens |
| | OX = 9606 GN = MICU1 |
| BCL7B_HUMAN | B-cell CLL/lymphoma 7 protein family member B OS = Homo sapiens |
| | OX = 9606 GN = BCL7B PE = 1 SV = 1 |
| BCL7B_HUMAN | Isoform 2 of B-cell CLL/lymphoma 7 protein family member B |
| | OS = Homo sapiens OX = 9606 GN = BCL7B |
| BCL7B_HUMAN | Isoform 3 of B-cell CLL/lymphoma 7 protein family member B |
| | OS = Homo sapiens OX = 9606 GN = BCL7B |
| BCL7B_HUMAN | Isoform 4 of B-cell CLL/lymphoma 7 protein family member B |
| | OS = Homo sapiens OX = 9606 GN = BCL7B |
| BIP_CORAV | Endoplasmic reticulum chaperone BiP OS = Corylus avellana OX = 13451 |
| | PE = 1 SV = 1 |
| ENO2_HEVBR | Enolase 2 OS = Hevea brasiliensis OX = 3981 GN = ENO2 PE = 1 SV = 1 |
| ENO1_HEVBR | Enolase 1 OS = Hevea brasiliensis OX = 3981 GN = ENO1 PE = 1 SV = 1 |

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for detecting the presence of an allergen in a food production chain, comprising:
obtaining sequence data for a plurality of nucleic acid sequences present in a food product;
identifying one or more allergen sequences in the sequence data, wherein the one or more allergen sequences correspond to an allergen present in the food sample; and
detecting the presence of the allergen in the food product if the one or more allergen sequences are above a predetermined threshold.

2. A method for detecting the presence of a toxigen in a food production chain, comprising:
obtaining sequence data for a plurality of nucleic acid sequences present in a food product;
identifying one or more toxigen sequences in the sequence data, wherein the one or more toxigen sequences correspond to a toxigen present in the food sample; and
detecting the presence of a toxigen in the food product if the one or more toxigen sequences are above a predetermined threshold.

3. The method of claim 1, wherein the predetermined threshold corresponds to the relative level of a sequence in the food sample or the sequence coverage of a sequence in the food sample.

4. (canceled)

5. The method of claim 1, wherein the allergen is of milk or egg origin.

6. The method of claim 2, wherein the toxigen is a toxigen produced by a bacteria, fungi or plant.

7. The method of claim 1, wherein obtaining the sequence data comprises preparing a sequencing library.

8. The method of claim 1, wherein obtaining the sequence data comprises next generation sequencing, or microarray analysis.

9. The method of claim 1, wherein the plurality of nucleic acid sequences are DNA sequences or RNA sequences.

10. (canceled)

11. The method of claim 9, wherein the RNA sequences correspond to mRNAs encoding polypeptides present in the food sample.

12. The method of claim 11, wherein the RNA sequences are converted into amino acid sequences corresponding to polypeptides encoded by the RNA sequences prior to identifying the one or more allergen sequences.

13. The method of claim 1, wherein sequences corresponding to the food product are filtered from the sequence data prior to identifying the one or more allergen sequences.

14. The method of claim 1, wherein sequences corresponding to microbes present in the product are filtered from the sequence data prior to identifying the one or more allergen sequences.

15. The method of claim 1, wherein:
identifying the one or more allergen sequences comprises comparing the sequence data against one or more databases of allergen sequences.

16. The method of claim 1, wherein:
the one or more databases of allergen sequences correspond to databases of allergen protein sequences.

17. The method of claim 1, wherein the method is performed at one or more points in a food production chain of the food product.

18. The method of claim 1, further comprising tracing the allergen to a particular supplier of the food product.

19. The method of claim 1, further comprising:
identifying the food product as allergenic if the allergen is detected in the food sample; and
adjusting a product label information for the food product to indicate that the food product is allergenic.

20. The method of claim 2, further comprising:
identifying the food product as toxigenic if the toxigen is detected in the food sample; and
removing the food product from the food production chain if the product is identified as toxigenic.

21. A system for detecting the presence of an allergen in a food production chain, comprising:
one or more processors; and
a memory comprising instructions executable by the one or more processors that, when executed by the one or more processors, cause the system to:
obtain sequence data for a plurality of nucleic acid sequences present in a food product;
identify one or more allergen sequences in the sequence data, wherein the one or more allergen sequences correspond to an allergen present in the food sample; and
detect the presence of the allergen in the food product if the one or more allergen sequences are above a predetermined threshold.

22. A system for detecting the presence of a toxigen in a food production chain, comprising:
one or more processors; and
a memory comprising instructions executable by the one or more processors that, when executed by the one or more processors, cause the system to:
obtain sequence data for a plurality of nucleic acid sequences present in a food product;
identify one or more allergen sequences in the sequence data, wherein the one or more allergen sequences correspond to a toxigen present in the food sample; and
detect the presence of a toxigen in the food product if the one or more toxigen sequences are above a predetermined threshold.
